(19) United States
(12) Patent Application Publication
(10) Pub. No.: US 2008/0027531 A1
(43) Pub. Date: Jan. 31, 2008

(54) Stent for Use in Cardiac, Cranial, and Other Arteries

(76) Inventors: Darrell H. Reneker, Akron, OH (US); Daniel J. Smith, Stow, OH (US); Woraphon Kataphinan, Fontana, CA (US)

Correspondence Address: ROETZEL AND ANDRESS, 222 SOUTH MAIN STREET, AKRON, OH, 44308, US

(21) Appl. No.: 10/597,901

(22) PCT Filed: Feb. 14, 2005
(86) PCT No.: PCT/US05/04532

§ 371 Date: Apr. 23, 2007

Related U.S. Application Data
(60) Provisional application No. 60/544,009, filed on Feb. 12, 2004.

(51) Int. Cl. A61F 2/06

(52) U.S. Cl. 623/1.15; 604/103.02; 977/931

(57) ABSTRACT

The present invention is directed to a medical device, such as a stent, having a coating comprising a release component and an insoluble fibrous component. The release component is capable of being degraded thus leaving a gap between the stent and the insoluble fibrous component. Further, the insoluble fibrous component is capable of being wrapped about the stent, and capable of moving substantially freely about the stent upon degradation of the release component. This capacity enables the insoluble fibrous component to form a reinforced thrombus plug in, for instance, an aneurysm or fistula.

This application claims priority of U.S. Provisional Patent Application Ser. No. 60/544,030, which is herein incorporated by reference in its entirety.

### BACKGROUND OF THE INVENTION

The coating of medical devices, including coating medical devices with fibrous coatings is known. For example, International Publication No. WO 02/49535A2 to Dubson et al. is directed to a medicated polymer-coated stent assembly. Dubson discloses using electrospinning for coating stents to obtain durable coating with wide range of fiber thickness and porosity. The pores are useful in delivering drugs or having the stent serve as a stent graft. To improve adhesion of the electrospun layer, Dubson discloses the use of adhesives and chemical binding. Greenhalgh et al. (U.S. Patent Application No. US 2003/0211135A1) discloses a stent having an electrospun covering of a fibrous polymer layer. The stent is covered with the fibrous polymer layer by providing a spinnerette charge with electric potential relative to a predetermine location on a target plate. The stent is placed between the spinnerette and the predetermined location on said target plate. The polymers are enforced through the spinnerette, thereby transferring at least some of the electric potential to the polymer such that the polymer forms a stream directed toward the target plate due to the electric potential between the liquid and the plate. Before it reaches the plate, the stream splays into a plurality of nanofibers due to the electric potential between the liquid and the plate. At least some, preferably most, of the nanofibers collide with the stent instead of reaching the target plate. The predetermined location on the target plate is then moved relative to the object until the entire object is covered. By heating the stent to a point where the fibrous, preferably electrospun, polymer loses its ability to span the gaps, the fibers spanning the gaps break and retract to the nearest wire by virtue of surface tension to produce a covered stent. Other electrostatically coated stents include U.S. Pat. Nos. 5,948,018; 5,723,004; and 5,639,278 to Dereume et al., U.S. Pat. No. 5,632,772 to Alcime et al., and U.S. Pat. No. 5,855,598 to Pinchuk.

Other coated medical devices, such as stents, include Hossainy et al. (Publication No. WO 03/082368A1) which discloses delivery of 40-O-(2-hydroxy) ethyl-rapamycin via a coated stent, wherein the coating can be achieved by spraying the composition or by immersing the prosthesis in the composition. Pathak et al. (Publication No. WO 03/035134A1) discloses stent coatings which include a combination of a restenosis inhibitor comprising an HMG-CoA reductase inhibitor and a carrier. The method for coating comprises blending a substantially unreacted HMG-CoA reductase inhibitor and a polymeric or non-polymeric carrier, and applying the coating composition to the stent by spraying the coating composition onto the stent, by immersing the stent in the coating composition, or by painting the stent with the coating composition. Shulze et al, (U.S. Patent Application No. 2003/0088307) is generally directed to a stent having a polymer coating applied as a coating by evaporating a solvent from a solution that has been applied to the stent surfaces. Sundar (U.S. Patent Application Publication No. 2003/0135255) is directed to a stent delivery system where the coating is applied rotationally while the body is at least partially immersed in a coating liquid. Other disclosures of coated devices include U.S. Patent Application Publication No. 2003/0190341 to Shalaby et al.,

U.S. Pat. No. 5,980,551 to Summers et al. is directed to a stent coated with a biodegradable, resorbable, and hemocompatible material. U.S. Pat. No. 6,569,195 to Yang et al. is directed to a stent having a polymeric coating for delivering a biologically active agent or other therapeutic substance over a target time. U.S. Pat. No. 6,627,246 to Mehta, et al. is directed to a process for coating stents and other medical devices with a film-forming biocompatible polymer and/or optional therapeutic agent using super-critical fluid deposition.

### SUMMARY OF THE INVENTION

The present invention is directed to a medical device, such as a stent, having a nanofibrous coating comprising a soluble, digestible, or otherwise degradable material and an insoluble nanofiber. Upon implantation, the degradable material component degrades in the subject's blood or other body fluid leaving behind a loose-fitting insoluble nanofiber. This loose-fitting fiber coating is sufficiently free-moving to be forced into an aneurysm or fistula under ordinary hydrostatic blood pressure, thus forming a partial plug or thrombogenic surface. Once inside the aneurysm or fistula, the nanofibrous partial plug acts as a thrombogenic surface for forming a nanofiber-reinforced thrombus plug, thus repairing the injury.

The present invention is directed to a stent comprising a stent member, and an external fibrous layer, wherein the layer is sufficiently loosely wrapped around the stent to allow the layer to deform in a manner that forms a reinforcing plug.

The present invention is also directed to a method for manufacturing a stent comprising the steps of coating a stent's external surface with a first release layer, and coating the outer surface of the first release layer with a second fibrous layer, wherein the first release layer is capable of being removed leaving the second fibrous layer sufficiently loosely wrapped around the stent to allow the second layer to deform in a manner that forms a reinforcing plug while remaining attached to the stent.

The present invention is also directed to a method for using a stent having an external fibrous layer that is loosely wrapped around the stent comprising the step of implanting the stent in a living organism.

The present invention is also directed to a balloon catheter comprising an external fibrous layer, wherein the layer is loosely wrapped around the balloon catheter.

The present invention is also directed to a method for manufacturing a balloon catheter having an external fibrous layer that is loosely wrapped around the balloon catheter comprising the steps of coating a balloon catheter's external surface with a first release layer, coating the outer surface of the first release layer with a second fibrous layer, and removing the first release layer thereby leaving the second fibrous layer loosely wrapped around the balloon catheter.

The following terms are specially defined. Loose, as used in the present application to describe the insoluble fibrous component, means sufficiently free-moving to allow the fibers to be forced into an aneurysm or fistula under ordinary hydrostatic blood pressure, thus forming at least a partial plug. The quality of being loose is not negated by the likelihood that the insoluble fibrous component may remain generally wound about the stent. The noun “opening” or “openings”, as used herein refers to aneurysms, fistulas, holes, gaps, fissures, through-holes, orifices, foramen, fenestrae, and the like. Particularly those which occur in arteries and veins. The term convoluted, as used herein to describe the conformation of the insoluble fibrous component, encompasses folded, wrinkled, corrugated, creased, crinkled, furrowed, plicaed, ridged, rimpled, riveted, rucked coiled, involuted, wound, twisted, spiraled, rolled, and entangled.

### BRIEF DESCRIPTION OF THE DRAWINGS

FIG. 1 is a drawing of a fiber wrapped helically about a stent

FIG. 2 is a drawing of a convoluted fiber wrapping about both sides of a stent

FIG. 3 is a drawing of an alternatively convoluted fiber wrapping about both sides of a stent

FIG. 4 is a drawing of another alternatively convoluted fiber wrapping about both sides of a stent

FIG. 5 is a drawing of a fiber sheet helically wrapping about a stent

FIG. 6 is a cross-sectional drawing of a stent having a first release layer with a second fibrous layer

FIG. 7 is a cross-sectional drawing of a stent having a co-deposited release component and fibrous component.

FIG. 8 is a drawing of a flared stent

FIG. 9 is a drawing of a flared stent implanted in a blood vessel and entrapping thrombogenic nanofibers which are shown to have flowed into an aneurysm.

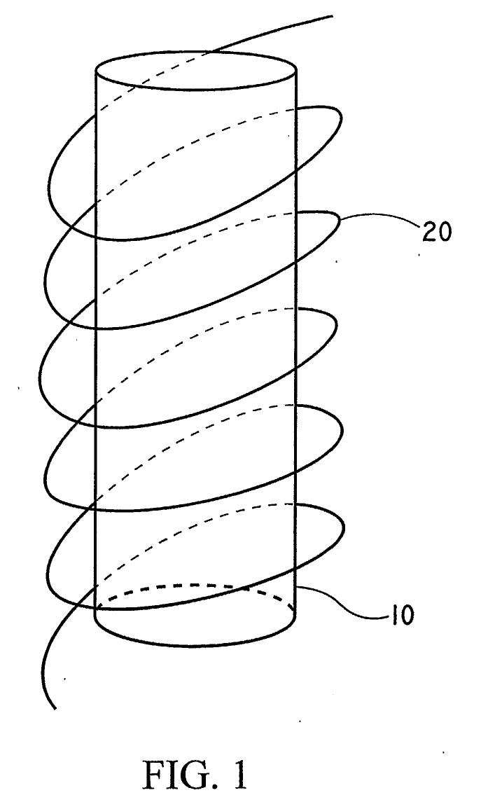

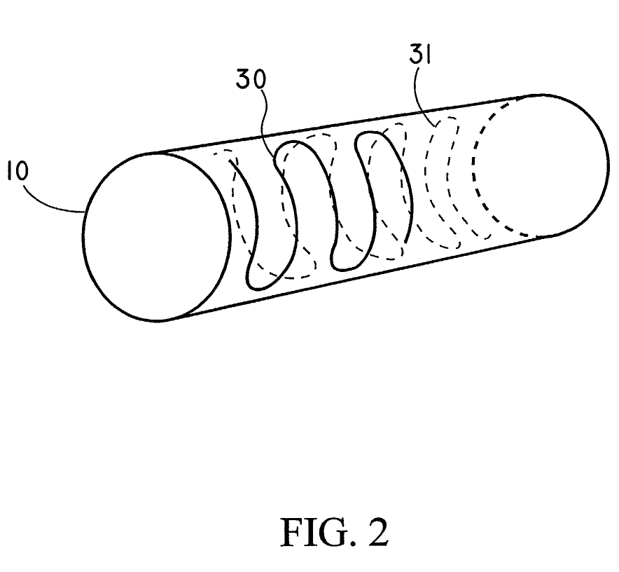

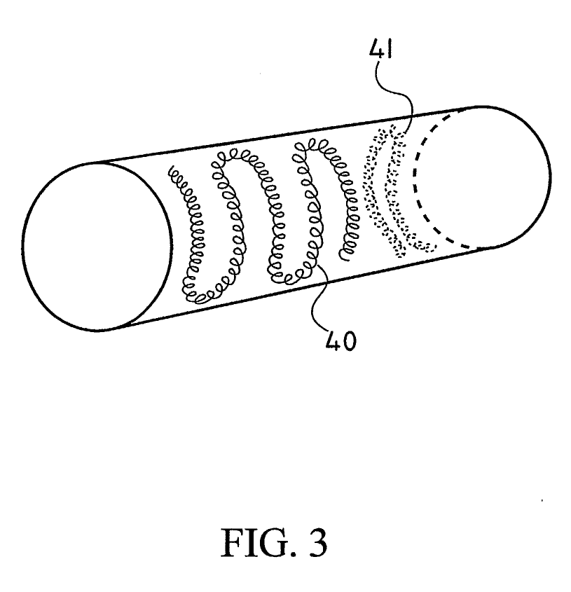

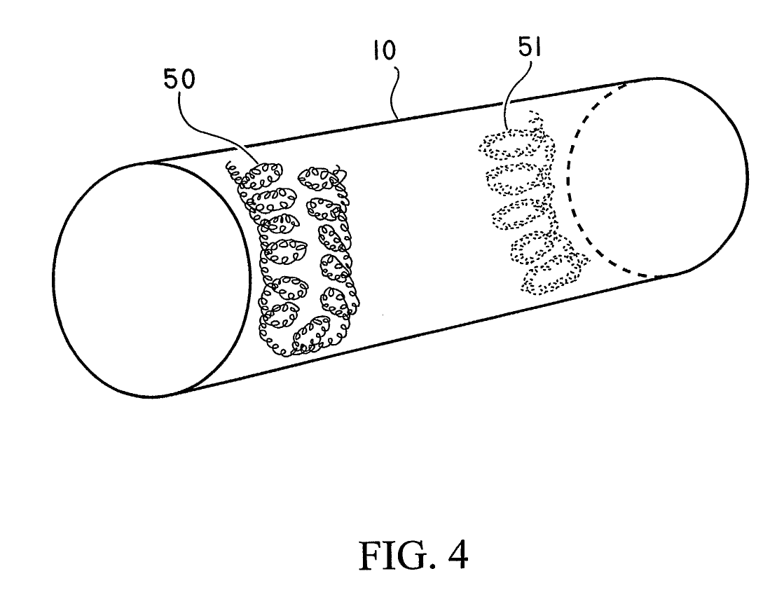

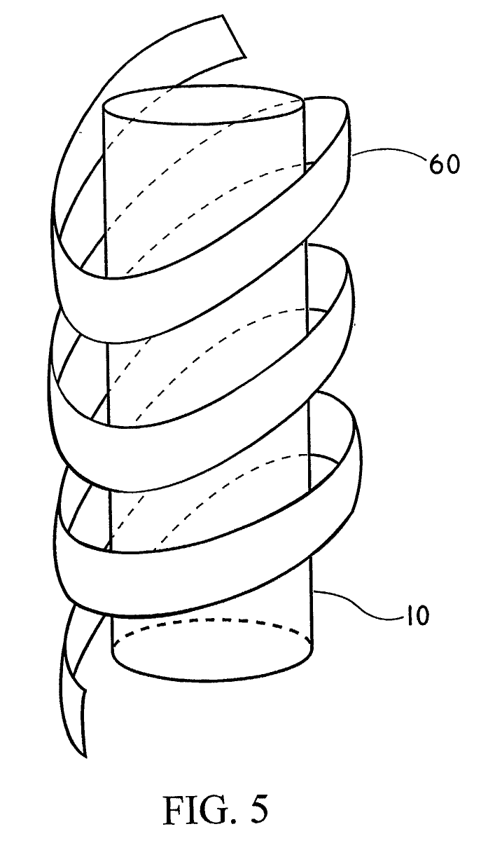

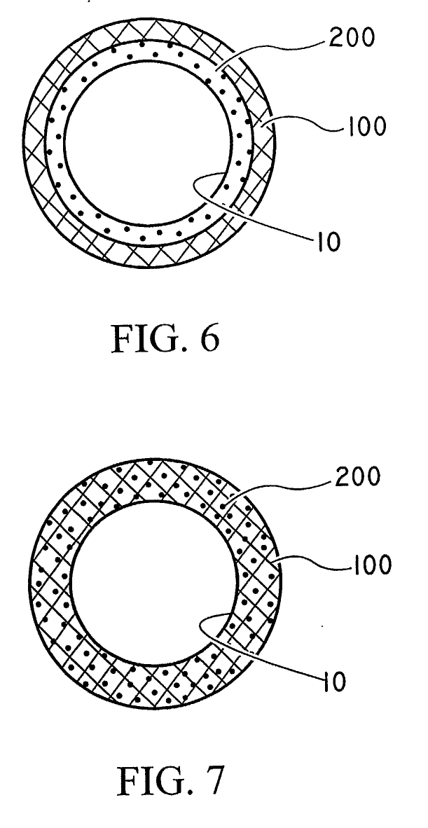

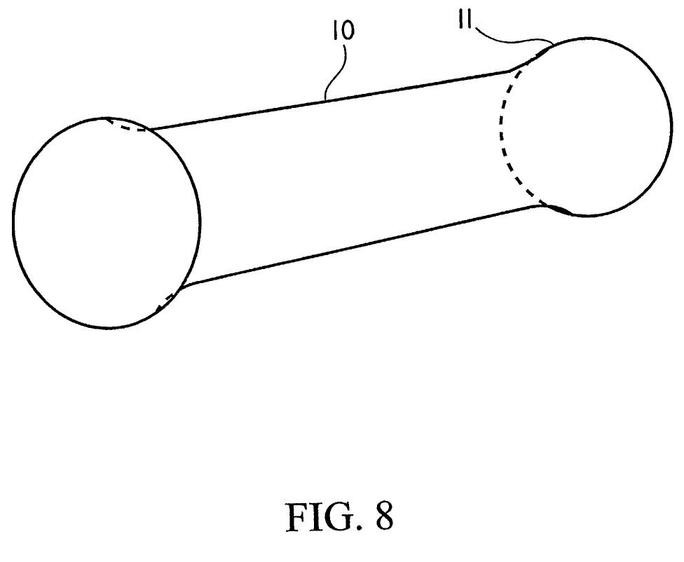

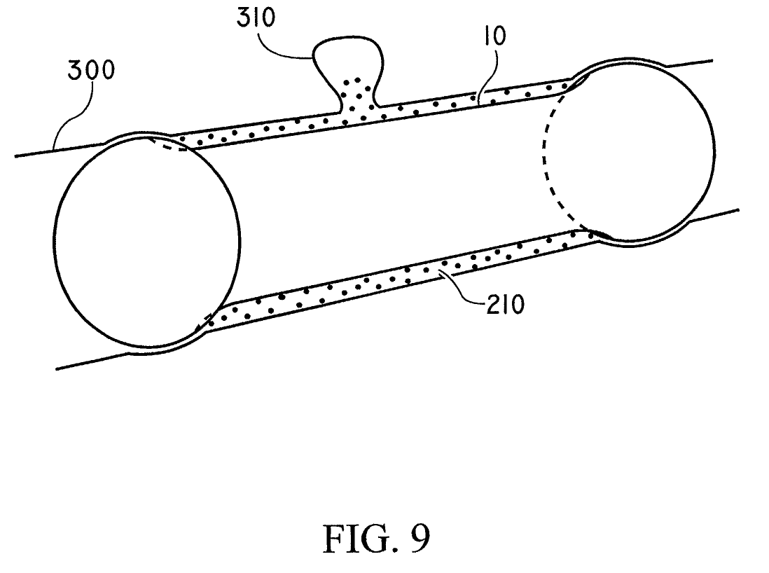

### DETAILED DESCRIPTION OF ILLUSTRATIVE EMBODIMENTS

The present invention is directed to a medical device, such as a stent, having a coating comprising a soluble, digestible, or otherwise degradable material (referred to hereinafter as a release layer or release component), an insoluble nanofiber, and an optional lubricant. Upon implantation, the degradable material component degrades in the subject's blood or other body fluid leaving behind a loose-fitting insoluble nanofiber. The degraded release component may serve as a lubricant for the insoluble fibrous component, which contributes to its substantially free motion; however, other suitable lubricants may include endogenous body fluids such as blood, or an optional lubricant additive. The loose-fitting fiber coating is sufficiently free-moving to be forced into an aneurysm, fistula, hole, gap, fissure, through-hole, orifice, foramen, fenestrae or other opening (herein after referred to collectively as “opening” or “openings”) under ordinary hydrostatic blood pressure, thus forming a partial plug or thrombogenic surface. Once inside the opening, the nanofibrous partial plug acts as a thrombogenic surface for forming a nanofiber-reinforced thrombus plug, thus repairing the injury.

Insoluble Fibrous Component

Suitable materials for forming fibers of the present invention include, but are not limited to poly(caprolactone), polyethylene terephthalate, fibrinogen, polyolefins, polyethylene, polypropylene, linear poly(ethylenimine), cellulose acetate, and other preferably grafted cellulosics, poly (L-lactic acid), poly (ethyleneoxide), poly (hydroxyethylmethacrylate), poly (glycolic acid) and poly vinylpyrrolidone. Poly(caprolactone) and polyethylene terephthalate are preferred. Other suitable materials include without limitation polyethylene glycol, polyethylene oxazoline, polyester, polyacrylic acid, polyacrylic acid esters, polyphosphezines, polycyanoacrylate, polyvinyl amines, polyethylene imines, polyethylene amines, polyacrylamides, cellulose, cellulose derivatives, proteins, polyorthoesters, polyanhydrides, polyketals, polyacetals, polyureas, and polycarbonate, or a combination thereof

Fibers of the present invention may be fabricated according to a variety of methods known in the art including electrospinning, wet spinning, dry spinning, melt spinning, and gel spinning. Electrospinning is particularly suitable for fabricating fibers of the present invention inasmuch as it tends to produce very thin (i.e. fine denier) fibers. Typically electrospun fibers can be produced having very small diameters, usually on the order of about 3 nanometers to about 3000 nanometers.

Another particularly effective method for producing nanofibers of the present invention comprises the nanofibers by gas jet method (i.e. NGJ method). This method has been previously described and is known in the art. Briefly, the method comprises using a device having an inner tube and a coaxial outer tube with a sidearm. The inner tube is recessed from the edge of the outer tube thus creating a thin film-forming region. Polymer melt is fed in through the sidearm and fills the empty space between the inner tube and the outer tube. The polymer melt continues to flow toward the effluent end of the inner tube until it contacts the effluent gas jet at the edge of the inner tube where it opens into the outer tube. The gas jet impinging on melt creates a thin film of polymer melt in the region between the edges of the inner and outer tubes, which travels to the effluent end of the outer tube where it is ejected forming a turbulent cloud of nanofibers.

Electrospinning and NGJ techniques permit the processing of polymers from both organic and aqueous solvents. Furthermore, it has been discovered that dispersions of discrete particles and soluble non-fiber forming additives into the fluid to be spun into the fiber (i.e., the spin dope) does not prevent the formation of fiber mats using electrospinning and NGJ techniques. Therefore a wide variety of additives may be incorporated into fibers and devices of the present invention. Accordingly, medicinal additives may be included such as antimicrobial and antibiotic drugs, and various other therapeutic agents.

Release Component

The release component generally comprises any biocompatible material that is capable of being coated on a stent and capable of being dissolved, digested or otherwise degraded. Although no particular degradation rate is preferred, a suitable rate is one that forms a gap, thus loosening the insoluble fiber component, in a timely manner tending to preserve the life and remediate the health of the subject. In general faster degradation rates tend to be better, but the rate should not be so fast that the stent cannot be implanted before degradation has reached a point where the insoluble nanofiber coating becomes capable of moving substantially independently of the stent, which could result in loss of the insoluble fibrous component. In some instances a longer degradation time may be suitable; for instance, several days or several weeks.

Suitable release components, preferably and without limitation, include polysaccharides, corn syrup, gelatin and collagen. Other suitable release component materials include without limitation peptides, nucleic acids especially ribonucleic acids, glycogen, and glycoproteins.

Release component materials may be coated onto the stent by any of a variety of methods known in the art including without limitation electrospinning, nanofibers by gas jet (NGJ), wet spinning, dry spinning and gel spinning. Suitable methods also include painting, spin coating, dipping, and spray coating. The release component may comprise a layer upon which the insoluble fibrous component sits, or it may comprise a matrix within which the insoluble fibrous component is entrained.

Degradation of release component materials may occur in any of a variety of biocompatible ways including without limitation dissolution by body fluids such as blood, or digestion by enzymes such as proteases, lipases, endonucleases, amylases, and the like. The source of the enzymes may be endogenous; an additive to the coating; a dietary, injectable or other supplement provided to the subject; or any other suitable source providing a bioactive enzyme to the release component. The degraded release component may serve as a lubricant for promoting the substantially free motion of the insoluble fibrous component.

Wrapping

Generally speaking, the insoluble fibrous component of the present invention is attached to the stent or medical device by wrapping the insoluble fibrous component about it. Wrapping serves two functions. First, it serves as a means of temporarily attaching the insoluble fibrous component to the stent until being released after implantation. Second, wrapping may, optionally, serve as an additional means of loosening the insoluble fibrous component after implantation. Recall that the other means of loosening is the use of a release component as mentioned above.

The principal of using wrapping patterns to cause the insoluble fibrous component to loosen upon implantation is essentially this: the wrapped insoluble fibrous component is temporarily bonded to the stent surface by the release component prior to implantation, but after implantation the release component is degraded and disappears, thus releasing the insoluble fibrous component from the surface. At this point the insoluble fibrous component is able to float substantially freely about the stent; it is still generally wound about the stent, but it is no longer bonded to the surface. Thus its range of motion is principally limited by the fact that it remains generally wrapped about the stent. At a minimum, the empty space left by the release component provides some range of motion for the insoluble fibrous component to float away from the stent. However, in addition to that, a convoluted insoluble fibrous component has a source of added range of motion, namely deconvoluting it. For instance, consider the relatively tight range of motion that results from a helically wrapped fiber versus the relatively loose range of motion that results from a convoluted fiber. In both cases the fiber obtains a range of motion by virtue of the degradation of the release component, but when the convoluted fiber is straightened it is has a greater reach than the helical alternative.

The insoluble fibrous component may be wrapped in any suitable pattern including but not limited to helical, helicoid, or any of various convoluted patterns. Wrapping may also be accomplished by randomly orienting the insoluble fibrous component wrap such that it has no apparent pattern. Electrospun and NGJ spun fibers often impinge the surface of a target substrate in disordered groups rather than straight lines. Therefore, a disordered fiber wrapping may be readily achieved by either method. Generally, the more convoluted the fiber the greater its capacity to loosen upon degradation of the release component.

Stents

The stent of the present invention serves as substrate, i.e. support, for an insoluble fibrous component and a release component. Accordingly, any stent presently known in the art is suitable for incorporation into the present invention provided it has the capacity to support the foregoing components. Additionally, the stent of the present invention preferably includes at least one and preferably two flared ends. The flares serve to entrap the loose insoluble fibrous component after the release component has been degraded. In principal, when the stent is expanded during implantation the flares contact the blood vessel preferentially relative to the body of the stent. Consequently, the flare forms a seal against the blood vessel wall, and leaves a void between the body of the stent and the blood vessel. This void contains the insoluble fibrous component. Thus the flares substantially prevent the insoluble fibrous component from oozing out of the void, and being lost in the blood stream.

### EMBODIMENTS

In one embodiment a stent is coated with a layer of release component material such as a polysaccharide. Then the stent is coated by, for instance, NGJ or electrospinning a layer of insoluble fibrous component such as polyethylene terephthalate. Thus this embodiment comprises two layers deposited on a substrate. Another embodiment is essentially the previous one, but the insoluble fibrous component is first electrospun into a free standing sheet, and then the sheet is applied to the stent. In yet another embodiment the release component and the insoluble fibrous component are co-deposited, for instance, by electrospinning or NGJ. Still another embodiment comprises any of the foregoing wherein the insoluble fibrous component is wrapped in a convoluted pattern. Still another embodiment includes an optional lubricant, which functions to lubricate the nanofibers thus allowing them to more readily be forced into an aneurysm or fistula. A lubricant could be added to the stent prior to implantation, or may be added through the same catheter as the stent during implantation, and may comprise without limitation mineral oil, or any biocompatible oil or grease. Yet another embodiment comprises any of the foregoing plus a medicinal additive.

In every embodiment of the present invention the thickness of the stent coating is on the order of millimeters. More particularly, the thickness comprises about five millimeters, but may be more or less depending on the size of the blood vessel being repaired. In principal a suitable thickness is determined by several factors including the size of the gap between the body of the stent and the blood vessel wall, the range of motion of the insoluble fibrous component, and the position of the blood vessel hole relative to the stent.

The foregoing embodiments of the present invention have been presented for the purposes of illustration and description. These descriptions and embodiments are not intended to be exhaustive or to limit the invention to the precise form disclosed, and obviously many modifications and variations are possible in light of the above disclosure. The embodiments were chosen and described in order to best explain the principle of the invention and its practical applications to thereby enable others skilled in the art to best utilize the invention in its various embodiments and with various modifications as are suited to the particular use contemplated. It is intended that the invention be defined by the following claims.

In order to demonstrate the practice of the present invention, the following examples have been prepared and tested. The examples should not, however, be viewed as limiting the scope of the invention. The claims will serve to define the invention.

What is claimed is:

1. A stent comprising:
a stent member; and
an insoluble fibrous component, wherein the component is sufficiently loosely wrapped around the stent to allow the component to deform in a manner that forms a reinforcing thrombus plug.

2. The stent of claim 1, wherein the insoluble fibrous component comprises at least one nanofiber.

3. The stent of claim 1, wherein the insoluble fibrous component comprises a compound selected from the group consisting of poly(caprolactone), polyethylene terephthalate, fibrinogen, polyolefins, polyethylene, polypropylene, linear poly(ethylenimine), cellulose acetate, grafted cellulosics, poly (L-lactic acid), poly (ethyleneoxide), poly (hydroxyethylmethacrylate), poly (glycolic acid) poly vinylpyrrolidone, polyethylene glycol, polyethylene oxazoline, polyester, polyacrylic acid, polyacrylic acid esters, polyphosphezines, polycyanoacrylate, polyvinyl amines, polyethylene imines, polyethylene amines, polyacrylamides, cellulose, polyorthoesters, polyanhydrides, polyketals, polyacetals, polyureas, and polycarbonate.

4. The stent of claim 1, wherein the insoluble fibrous component comprises a thrombogenic material that initiates the formation of a thrombus.

5. The stent of claim 4, wherein the thrombogenic material at least partially blocks the entrance to a structure selected from the group consisting of an aneurysm, a fistula, and an opening in a blood vessel wall.

6. A method for manufacturing a stent comprising the steps of:
coating a stent with a release layer; and
coating the release layer with an insoluble fibrous layer, wherein the release layer is capable of being degraded leaving the insoluble fibrous layer sufficiently loosely wrapped around the stent to allow the insoluble fibrous layer to deform and move in a manner that allows it to form a reinforcing plug.

7. The method of claim 6, wherein the release layer is soluble in blood the fibrous layer is insoluble in blood.

8. The method of claim 6, wherein the release layer is capable of being digested by enzymes.

9. The method of claim 6, wherein the release layer comprises a material selected from the group consisting of polysaccharides, corn syrup, gelatin, collagen, peptides, proteins, nucleic acids, and ribonucleic acids.

10. The method of claim 6, wherein the insoluble fibrous layer comprises a thrombogenic material.

11. The method of claim 10, wherein the thrombogenic material is selected from the group consisting of poly(caprolactone), polyethylene terephthalate, fibrinogen, polyolefins, polyethylene, polypropylene, linear poly(ethylenimine), cellulose acetate, grafted cellulosics, poly (L-lactic acid), poly (ethyleneoxide), poly (hydroxyethylmethacrylate), poly (glycolic acid) poly vinylpyrrolidone, polyethylene glycol, polyethylene oxazoline, polyester, polyacrylic acid, polyacrylic acid esters, polyphosphezines, polycyanoacrylate, polyvinyl amines, polyethylene imines, polyethylene amines, polyacrylamides, cellulose, polyorthoesters, polyanhydrides, polyketals, polyacetals, polyureas, and polycarbonate.

12. The method of claim 6, wherein the release layer comprises a nanofiber.

13. The method of claim 6, wherein the insoluble fibrous layer comprises a nanofiber.

14. The method of claim 6, wherein the steps of coating the stent comprises electrospinning.

15. The method of claim 6, wherein the step of coating the release layer with an insoluble fibrous layer comprises electrospinning.

16. A method for using the stent of claim 1 comprising the step of implanting the stent in a living organism.

17. A balloon catheter comprising:
an insoluble fibrous layer, wherein the layer is capable of becoming loosely wrapped around the balloon catheter upon degradation of a release component.

18. The balloon catheter of claim 17, wherein the insoluble fibrous layer comprises a nanofiber.

19. The balloon catheter of claim 17, wherein the external fibrous layer comprises polyethyleneoxide, polyethylene glycol, polyethylene oxazoline, polyester, polycaprolactone, polyacrylic acid, polyacrylic acid esters, polyhydroxyethylmethacrylate, polyvinyl pyrollidone, polyphosphezines, polycyanoacrylate, polyvinyl amines, polyethylene imines, polyethylene amines, polyacrylamides, cellulose, cellulose derivatives, proteins, polyorthoesters, polyanhydrides, polyketals, polyacetals, polyureas, and polycarbonate, or a combination thereof.

20. The balloon catheter of claim 17, wherein the external layer comprises a thrombogenic material that initiates the formation of a thrombus.

21. The balloon catheter of claim 20, wherein the thrombogenic material at least partially blocks the entrance to an aneurysm or an opening in a blood vessel wall.

22. A method for manufacturing a balloon catheter having an external fibrous layer that is loosely wrapped around the balloon catheter comprising the steps:
coating a balloon catheter's external surface with a release layer;
coating the outer surface of the release layer with a fibrous layer; and
removing the release layer thereby leaving the fibrous layer loosely wrapped around the balloon catheter.

23. The method of claim 22, wherein the release layer is soluble and the fibrous layer is insoluble in a liquid.

24. The method of claim 22, wherein the release layer can be degraded to a soluble or gaseous species by enzymes, small molecules, or other reactive substances.

25. The method of claim 22, wherein the release layer comprises polyethyleneoxide, polyethylene glycol, polyethylene oxazoline, polyester, polycaprolactone, polyacrylic acid, polyacrylic acid esters, polyhydroxyethylmethacrylate, polyvinyl pyrollidone, polyphosphezines, polycyanoacrylate, polyvinyl amines, polyethylene imines, polyethylene amines, polyacrylamides, cellulose, cellulose derivatives, proteins, polyorthoesters, polyanhydrides, polyketals, polyacetals, polyureas, and polycarbonate, or a combination thereof.

26. The method of claim 22, wherein the fibrous layer comprises a thrombogenic agent.

27. The method of claim 26, wherein the thrombogenic agent is fibrinogen, collogen, or a combination thereof.

28. The method of claim 22, wherein the release layer comprises a nanofiber.

29. The method of claim 22, wherein the fibrous layer comprises a nanofiber.

30. The method of claim 22, wherein the step of coating the balloon catheter's external surface comprises electrospinning.

31. The method of claim 22, wherein the step of coating the outer surface of the release layer with a fibrous layer comprises electrospinning.

32. A method for using a balloon catheter having an external fibrous layer that is loosely wrapped around the balloon catheter comprising the step of implanting the balloon catheter in a living organism.

33. A method for manufacturing a stent comprising the steps of:
Simultaneously coating a stent's external surface with a release component and an insoluble fibrous component, wherein the release component is capable of being degraded leaving the insoluble fibrous component sufficiently loosely wrapped around the stent to allow the insoluble fibrous layer to deform and move in a manner that forms a reinfored thrombus plug.

34. The method of claim 33, wherein the release component is soluble and the insoluble fibrous layer is insoluble in blood.

35. The method of claim 33, wherein the insoluble release layer can be degraded by enzymes.

36. The method of claim 33, wherein the release layer comprises a compound selected from the group consisting of polysaccharides, corn syrup, gelatin, collagen, peptides, ribonucleic acids, deoxyribonucleic acids, glycogen, and glycoproteins.

37. The method of claim 33, wherein the insoluble fibrous component comprises a thrombogenic material.

38. The method of claim 37, wherein the thrombogenic material is fibrinogen, collagen, or a combination thereof.

39. The method of claim 33, wherein the insoluble release component comprises a nanofiber.

40. The method of claim 33, wherein the insoluble fibrous component comprises a nanofiber.

41. The method of claim 33, wherein the step of coating the stent comprises a method selected from the group consisting of electrospinning and nanofibers by gas jet.

42. The method of claim 33, wherein the step of coating the release component with an insoluble fibrous component comprises a method selected from the group consisting of electrospinning and nanofibers by gas jet.
